(19) United States
(12) Patent Application Publication
(10) Pub. No.: US 2020/0178816 A1
(43) Pub. Date: Jun. 11, 2020

(54) SYSTEM FOR DETERMINING CORONARY PULSE WAVE VELOCITY

(71) Applicants: HOSPICES CIVILS DE LYON, LYON Cedex 2 (FR); UNIVERSITÉ CLAUDE BERNARD LYON 1, VILLEURBANNE (FR)

(72) Inventors: Pierre LANTELM, FONTAINES SAINT MARTIN (FR); Brahim HARBAOUI, BRON (FR); Andrei CIVIDJIAN, MEYZIEU (FR)

(21) Appl. No.: 16/306,191

(22) PCT Filed: May 30, 2017
(86) PCT No.: PCT/EP2017/063020

§ 371 Date: Nov. 30, 2018

(30) Foreign Application Priority Data
Jun. 2, 2016 (EP) 16305641.9

(51) Int. Cl. A61B 5/021; A61B 5/0215; G08B 21/02

(52) U.S. Cl. CPC A61B 5/02125; G08B 21/02; A61B 5/02158; A61B 5/02116

(57) ABSTRACT

An apparatus for determining coronary pulse wave velocity uses proximal and distal pressure sensors that are separated by a known distance to collect waveforms with rising and falling edges and to use times between those edges together with the known distance to determine coronary pulse wave velocity.

### RELATED APPLICATIONS

This is the national stage, under § 371, of international application PCT/EP2017/063020, filed on May 30, 2017, which claims the benefit of the Jun. 2, 2016 priority date of European Application 16305641.9.

### FIELD OF INVENTION

The invention relates to systems for assisting in the study of heart disease, and in particular to systems making it possible to predict a risk of an intracoronary plaque rupturing.

### BACKGROUND

It is known that the calcification hardens an artery. Vascular hardening is recognized as playing an important role in vascular accidents. Medical studies have suggested that the pulse wave velocity in an arterial segment is reasonably representative of such hardening. Studies have also confirmed that hardening of the aorta, measured by the pulse wave velocity thereof, is also an indicator allowing coronary disease to be predicted.

Although there exist systems allowing aortic pulse wave velocities to be measured, and hence corresponding studies to be performed, there exists no system allowing a coronary pulse wave velocity to be accurately determined. Practitioners therefore do not have access to any system allowing them to measure coronary pulse wave velocity and thus to determine the effect of coronary hardening on the progression of a coronary lesion, such as for example the risk of acute thrombosis. Furthermore, determining the aortic pulse wave velocity has proven to be insufficient for accurately determining disease in coronary arteries. In particular, measuring the aortic pulse wave velocity does not make it possible to predict a risk of an intracoronary plaque rupturing.

### SUMMARY

The invention thus pertains to a system for determining coronary pulse wave velocity. Such a system includes an interface and a processing device. The interface receives a proximal blood pressure signal from a coronary artery and receives a distal blood pressure signal from the coronary artery. The processing device is one that is configured to identify the start time Tfmp of a rising edge and the start time Tfdp of a falling edge on the basis of a received proximal pressure signal; to calculate the duration T between the identified times Tfdp and Tfmp; to identify the start time Tfdd of the falling edge from a received distal pressure signal; to identify the start time Tfmd of the rising edge of the distal pressure signal, after having subtracted said calculated duration T from the start time Tfdd of the falling edge of the distal pressure signal; to retrieve the value of the distance between the proximal pressure measurement location and the distal pressure measurement location; to calculate the duration separating the start of the respective identified rising edges of the proximal pressure signal and of the distal pressure signal; and to calculate the coronary pulse wave velocity according to said duration separating the rising edges and according to said retrieved distance value.

In some embodiments, the processing device is configured to calculate the derivative of the distal blood pressure signal, and is configured to identify Tfmd as corresponding to the time of the peak of the calculated derivative that is closest to the time defined by Tfdd−T.

In other embodiments, the processing device is configured: to derive a distal blood pressure signal PSCD₂ by subtracting a distal diastolic pressure from the received distal blood pressure signal; to derive a proximal blood pressure signal PSCP₂ by subtracting a proximal diastolic pressure from the received proximal blood pressure signal; to synchronize the signals PSCD₂ and PSCP₂ on the start of their respective falling edges; and to calculate a measure of similarity between the signals PSCD₂ and PSCP₂ over the duration T.

In yet other embodiments, the processing device is configured to determine Tfmd in an interval defined by the following inequality:

Tfdd−1.1*T<Tfmd<Tfdd−0.9*T.

In still other embodiments, the processing device determines the time Tfmd at the intersection between a distal diastolic pressure and a tangent to a rising edge of a distal pressure signal.

Also among the embodiments are those in which the receiving interface is configured to receive an electrocardiogram signal, and the processing device is configured to calculate the durations separating a QRS complex of the electrocardiogram signal from the time Tfmp and from the time Tfmd, respectively.

Still others of these embodiments have the processing device determining the distal diastolic pressure by calculating the intersection between: a polynomial or logarithmic function that is adjusted for the distal pressure signal before the start time of the QRS complex and extrapolated after the start time of the QRS complex and the tangent to the rising edge of the distal pressure.

In some of the embodiments, the processing device determines the time Tfmp at the intersection between a tangent to the rising edge of the proximal pressure signal and a proximal diastolic pressure, and wherein the processing device determines the time Tfdp at the intersection between a tangent to the falling edge of the proximal pressure signal and a proximal systolic pressure.

The set of embodiments also includes those in which the processing device is configured to calculate the durations separating a QRS complex of the electrocardiogram signal from the time Tfmp and from the time Tfmd, respectively, by: creating a first reconstructed signal that is equal to zero everywhere except for a period having a width of 10-20 ms and centered on the time corresponding to the rising edge of the blood pressure Tfmp during which the reconstructed signal is equal to a constant, to a triangular function or to the amplitude of the QRS complex of the electrocardiogram signal; creating a second reconstructed signal that is equal to zero everywhere except for a period having a width of 10-20 ms and centered on the time corresponding to the rising edge of the blood pressure Tfmd during which the reconstructed signal is equal to a constant, to a triangular function or to the amplitude of the QRS complex of the electrocardiogram signal; calculating the intercorrelation function between the electrocardiogram signal and the first reconstructed signal over the duration of a cardiac cycle or over the duration of a respiratory cycle; calculating the intercorrelation function between the electrocardiogram signal and the second reconstructed signal over the duration of a cardiac cycle or over the duration of a respiratory cycle; and calculating the mean, over a cardiac or respiratory cycle, of the duration separating a QRS complex of the electrocardiogram signal from the time Tfmp and from the time Tfmd, respectively, on the basis of the offset between the electrocardiogram and the reconstructed signals for which the intercorrelation function is maximum, if and only if the value of this maximum is above a threshold that is preferably between 0.4 and 0.6.

However, there exist yet other embodiments, among which are those in which, if the calculation of the measure of similarity between the signals PSCD₂ and PSCP₂ over the duration T is insufficient, the processing device calculates a measure of similarity between the signals PSCD₂ and PSCP₂ over an interval between 0.2*T and 0.8*T of the synchronized signals, with various time offset values, determines for which time offset Tajust this measure of similarity between the signals PSCD₂ and PSCP₂ over this interval is maximum, and determines Tfmd=Tfdd−T+Tajust.

There are also embodiments such that, if the calculation of the measure of similarity between the signals PSCD₂ and PSCP₂ over the duration T is insufficient, the processing device: retrieves a proximal blood pressure signal between the times Tfmp and Tfdp; adapts the retrieved signal to the scale of the distal blood pressure signal; replaces the distal blood pressure signal between the times Tfdd−T and Tfdd with the retrieved and adapted pressure signal; and determines Tfmd as the intersection between the tangent to the adapted retrieved signal and a distal diastolic pressure.

Further embodiments include those in which the processing device: determines the value of the distal blood pressure signal at the time Tpfm=Tfmd−Tana, where Tana is between 5 and 10 ms; calculates the ratio of the blood pressure value determined at the time Tpfm to a distal diastolic blood pressure; and generates a warning signal if the calculated ratio is higher than a threshold between 0.95 and 1.05.

In addition to the embodiments already elucidated, there also exist embodiments in which the system further comprises an elongated FFR guide wire including two pressure sensors that are separated by a predefined distance along the guide wire, the two pressure sensors being connected to the receiving interface, the receiving interface comprising a circuit for sampling the respective signals from the pressure sensors. Among these are embodiments in which this predefined distance between the two pressure sensors is between seventy and one hundred and fifty millimeters.

The embodiments described herein represent only non-abstract embodiments in which a technological improvement has been achieved by providing a way to measure coronary pulse wave velocity. The embodiments interact with matter in order to take measurements of certain physical quantities and to take action based on those measurements. As used herein, and taking full advantage of Applicant's right to be his own lexicographer, the term “non-abstract” shall be construed to mean and to only mean subject matter that is statutory subject matter within the meaning of 35 USC 101 as of the filing date of this application. The claims cover only non-abstract implementations of an apparatus and only non-abstract practices of methods. All abstract implementations of apparatus and methods are hereby disclaimed.

Other characteristics and advantages of the invention will become clearly apparent from the description that is given below thereof by way of indication and without any limitation with reference to the appended drawings, in which:

### BRIEF DESCRIPTION OF THE FIGURES

FIG. 1 is a schematic representation of a heart and of its coronary arteries;

FIG. 2 is a sectional view of a guide wire according to one aspect of the invention inserted into a coronary artery including a stenosis;

FIG. 3 is a schematic sectional view of an FFR guide wire device according to one aspect of the invention;

FIG. 4 is a schematic representation of a signal-processing system for the purpose of determining the pulse wave velocity and the ischemic nature of a coronary stenosis according to one aspect of the invention;

FIG. 5 is a diagram illustrating an example of the proximal coronary blood pressure PSCP, with a corresponding electrocardiogram signal, the derivative of this pressure PSCP, and a signal reconstructed on the basis of the electrocardiogram signal (SPR), as described in detail below;

FIG. 6 is a diagram illustrating an example of the distal coronary blood pressure PSCD, with a corresponding electrocardiogram signal, the derivative of this pressure PSCD, and a signal reconstructed on the basis of the electrocardiogram signal (SDR), as described in detail below;

FIG. 7 illustrates an example of the processing performed on the pressure signal PSCP;

FIG. 8 illustrates an example of the processing performed on the pressure signal PSCD;

FIGS. 9 and 10 illustrate the selection of a portion of the signal PSCP;

FIGS. 11 and 12 illustrate the use of a portion of the signal PSCP to determine a rising edge of the signal PSCD; and

FIG. 13 is a detailed view of a distal coronary blood pressure PSCD diagram illustrating a particular case of determining the extrapolated diastolic pressure (PDE).

### DETAILED DESCRIPTION

The invention makes it possible to accurately and reproducibly determine the coronary pulse wave velocity (and potentially the degree of stenosis of the coronary lesion), thus facilitating the decision-making process of the practitioner with a view to determining patient care. Additionally, the invention may be implemented at the same time as the FFR guide wire insertion procedure, which has already been clinically approved.

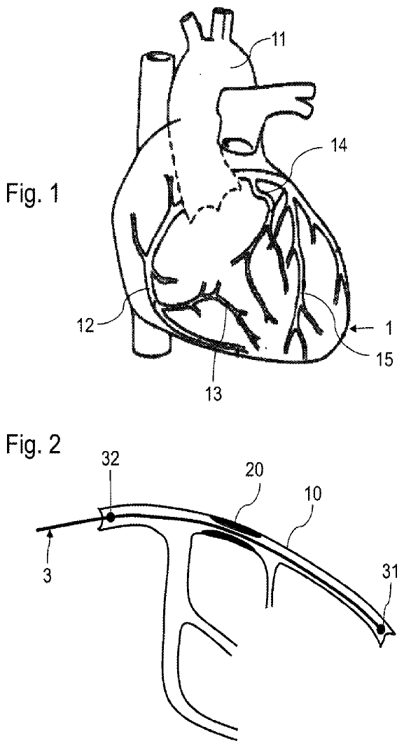

FIG. 1 is a schematic representation of a human heart 1. The aortic artery 11 connected to the heart and coronary arteries 12-15 are discernible. FIG. 1 illustrates the right coronary artery 12, the posterior descending coronary artery 13, the left circumflex coronary artery 14, and the left anterior descending coronary artery 15, all of which supply oxygenated blood to the heart muscles.

FIG. 2 illustrates an example of a method for retrieving signals to calculate a patient's coronary pulse wave velocity. The method includes inserting an FFR guide wire 3 so as to position its free end inside a coronary artery 10. The guide wire 3 includes first and second pressure sensors 31, 32 at its free end. The first pressure sensor 31 is in the distal position in order to measure the blood pressure in proximity to the junction of the artery 10 with the heart. The second pressure sensor 32 is in the proximal position in order to measure the blood pressure in proximity to the junction of the artery 10 with the aortic artery. The second pressure sensor 32 is separated from the first pressure sensor 31 by a predefined distance Dmd along the guide wire 3. The coronary artery 10 illustrated herein includes a stenosis 20 between the first and second pressure sensors 31, 32.

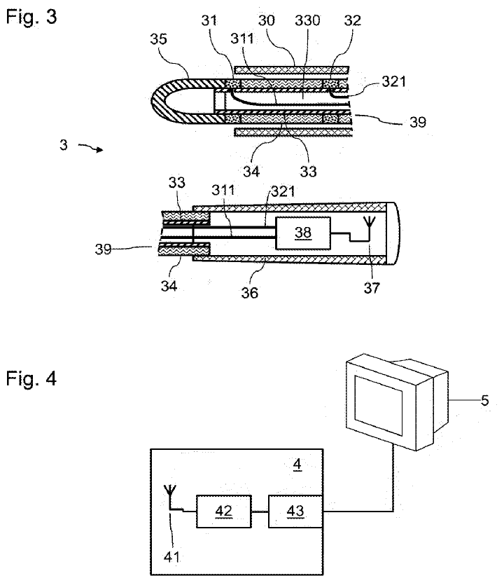

FIG. 3 is a schematic sectional view of two ends of a guide wire 3. The guide wire 3 includes a sliding wire 39 that slides through an outer storage sheath 30. The sliding wire 39 is only illustrated schematically in order to show its structure. The sliding wire 39 is not illustrated to scale. Because it is flexible, the sliding wire 39 adapts to the morphology of the coronary artery into which it is inserted. The sliding wire 39 includes a hollow metal sleeve 33. The metal sleeve 33 is covered with a sheath 34 made of synthetic material. In some embodiments, the sliding wire 39 includes a tip 35 at its free end. Among these are embodiments in which the tip 35 is flexible and radiopaque. In the illustrated embodiments, the tip 35 is attached to the metal sleeve 33.

The first pressure sensor 31 attaches to the periphery of the sleeve 33′ between the tip 35 and the sheath 34 to measure the distal blood pressure. The first pressure sensor 31 connects to a cable 311 for transmitting the pressure signal. The cable 311 passes through an aperture made in the sleeve 33 for connection to the first pressure sensor 31. The wire 311 extends through an inner bore 330 of the sleeve 33.

The second pressure sensor 32 attaches to the periphery of the sleeve 33 between two segments of the sheath 34 to measure the proximal blood pressure. The second pressure sensor 32 connects to a cable 321 for transmitting the pressure signal. The cable 321 passes through an aperture made in the sleeve 33 for connection to the second pressure sensor 32. The wire 321 extends through the inner bore 330 of the sleeve 33.

The sliding wire 39 is flexible but substantially incompressible or inextensible. Thus, the sliding wire 39 maintains a constant distance Dmd between the first and second pressure sensors 31, 32. The distance between the sensors 31 and 32 corresponds in practice to the curvilinear distance between these sensors along the sliding wire 39. The distance between the first and second pressure sensors 31, 32 is advantageously at least equal to seventy millimeters so as to ensure that the distance between the first and second pressure sensors 31, 32 is great enough to provide a high level of accuracy for the pulse wave velocity calculation. In some embodiments, the distance between the first and second pressure sensors 31, 32 is at most equal to one hundred and fifty millimeters such that the guide wire 3 can still be used in the majority of coronary arteries of standard length. Furthermore, using a guide wire 3 having first and second pressure sensors 31, 32 that are held at a predefined distance makes it possible to exclude inaccuracies due to the distance between two pressure measurements inside the same coronary artery.

Opposite its free end, the sliding wire 39 attaches to a handle 36. The sleeve 33 and the sheath 34 are embedded in the handle 36. The handle 36 thus allows the sliding wire 39 to be moved. In this example, the guide wire 3 is configured to deliver the measured pressure signals to a processing system via a wireless interface. However, it is also possible to envisage the guide wire 3 communicating with a processing system via a wired interface.

A drive circuit 38 is housed inside the handle 36. The cables 311 and 321 of the sliding wire 39 are connected to the drive circuit 38. The drive circuit 38 is connected to a transmitting antenna 37. The drive circuit 38 is configured to digitize the signals measured by the first and second pressure sensors 31, 32 and delivered by the cables 311 and 321 and to then to transmit the digitized signals remotely, via the antenna 37, using a suitable communication protocol.

In some embodiments, the sheath 34 is made of a hydrophobic material at the free end of the sliding wire 39. In other embodiments, the sheath 34 is made of another material, such as PTFE, between the free end and the handle 36.

The guide wire 3 communicates with a signal-processing system 4. The system 4 comprises here a wireless communication interface 41 for communicating with the guide wire 3. The system 4 thus comprises a receiving antenna 41 that is configured to receive the information communicated by the antenna 37. The receiving antenna 41 is connected to a processing circuit 42. The system 4 comprises a wired communication interface 43. The interface 43 allows for example the results calculated by the processing circuit 42 to be displayed on a display screen 5. An analog-to-digital converter may for example be incorporated within the processing circuit 42, namely within the guide wire 3, in order to allow the processing circuit 42 to process digital proximal and distal coronary blood pressure signals.

The processing circuit 42 is configured to identify the start time Tfmp of a rising edge in the proximal coronary blood pressure signal (referred to by PSCP hereinafter) received over the interface 41; to identify the start time Tfdp of a falling edge in the pressure signal PSCP received over the interface 41; to calculate the duration T between the times Tfdp and Tfmp that have been identified; to identify the start time Tfdd of a falling edge in the distal coronary blood pressure signal (referred to by PSCD) received over the interface 41; to identify the start time of a rising edge in the pressure signal PSCD received over the interface 41 by subtracting the duration T from the time Tfdd. A time Tfmd will be selected for example such that Tfdd−1.1*T<Tfmd<Tfdd−0.9*T; to retrieve the value of the distance Dmd between the proximal pressure measurement location and the distal pressure measurement location. This distance Dmd is for example stored in the guide wire 3 or in the system 4 when the guide wire 3 includes the first and second pressure sensors 31, 32 that are spaced apart by a predetermined distance; to calculate the duration Tmd separating the start of the rising edges Tfmp and Tfmd; and to calculate the coronary pulse wave velocity on the basis of this duration Tmd and of the distance Dmd.

In some embodiments, a signal PSCD₂ is derived by subtracting the distal diastolic pressure from the signal PSCD (the distal diastolic pressure will be described in detail below), a signal PSCP₂ is derived by subtracting the proximal diastolic pressure from the signal PSCP (the proximal diastolic pressure will be described in detail below), the signals PSCP₂ and PSCD₂ are synchronized on the starts of their falling edges, then a measure of similarity between the signals PSCP₂ and PSCD₂ for a duration equal to T is calculated. The measure of similarity may be calculated with a mean squared deviation between the signals PSCP₂ and PSCD₂ or by means of an autocorrelation function between these signals. The calculation of the measure of similarity between the signals PSCP₂ and PSCD₂ for a duration equal to T and for a plurality of offsets between PSCP₂ and PSCD₂ is repeated in the time interval [−0.1*T; 0.1*T], with time increments that are for example between 0.1 milliseconds and 0.5 milliseconds (corresponding to the sampling period), over a window of ±20 milliseconds. The optimum offset for greatest similarity between PSCP₂ and PSCD₂ (Tajust) is calculated.

Next, the steps of identifying the time Tfmd are carried out by subtracting the duration T from the time Tfdd and by adding Tajust, the duration Tmd is calculated, the value of the distance Dmd is retrieved, and the coronary pulse wave velocity is calculated as described in detail above, by dividing Dmd by Tmd.

Practically, it can be observed that the pressure signal PSCP is very faithful to the aortic pressure. The pressure signal PSCP thus includes fewer artifacts that are able to alter the result of digital processing operations to which it is subjected. Conversely, in particular because of blood microcirculatory effects, the pressure signal PSCD may include a large number of artifacts that are able to alter the result of digital processing operations to which it is subjected. In particular, the rising edge of the pressure signal PSCD has proven to be particularly sensitive to such artifacts, directly determining the start of this rising edge by analyzing this rising edge alone proving to be particularly inaccurate. Conversely, the inventors have observed that the falling edge of the pressure signal PSCD is relatively unaffected by artifacts. Consequently, the method determines the start of the rising edge of the pressure signal PSCD (or PSCD₂) by determining the start of the falling edge of the pressure signal PSCD, then by retracing back from there to the start of the rising edge of the pressure signal PSCD on the basis of the duration separating the accurately determined rising and falling edges for the pressure signal PSCP (or PSCP₂).

FIG. 7 illustrates an example of the processing performed on the pressure signal PSCP. In this example, the processing circuit 42 determines: the tangent 61 (illustrated by a dashed line) to the pressure PSCP rising edge; the diastolic pressure illustrated by the straight line 62 (illustrated by a dotted line); the tangent 63 (illustrated by a solid line) to the pressure PSCP falling edge; and the systolic pressure illustrated by the straight line 64 (illustrated by a dotted line).

The processing circuit 42 determines here the start time Tfmp of the rising edge of the pressure PSCP via the abscissa of the intersection between the tangent 61 and the diastolic pressure straight line 62. The processing circuit 42 determines here the start time Tfdp of the rising edge of the pressure PSCP via the abscissa of the intersection between the tangent 63 and the systolic pressure straight line 64.

Other methods for determining the times Tfmp and/or Tfdp may also be implemented by the processing circuit 42. It is possible for example to calculate the first or second derivative of the pressure PSCP, then to determine the times at which this first or second derivative crosses a positive threshold and a negative threshold, respectively (in order to identify a positive peak and a negative peak, respectively). These times may be used for the values Tfmp and Tfdp, respectively.

The systolic pressure and the diastolic pressure may be calculated in a manner known per se, by analyzing the pressure PSCP over at least one complete heartbeat, by taking for example the extrema of the pressure PSCP.

Advantageously, before processing the signals PSCP (or PSCP₂) or PSCD (or PSCD₂), the circuit 42 may implement low-pass filtering (for example at a cutoff frequency of between 10 and 20 Hz) in order to remove the rapid pressure fluctuations between heartbeats.

In some embodiments, as illustrated in FIG. 13 for the distal blood pressure (but also applicable to the proximal blood pressure), the diastolic pressure used in the calculations will be determined by extrapolation: Specifically, the time Tqrs just before the contraction of the heart (QRS complex) is determined; a polynomial or logarithmic function that is adjusted for the blood pressure samples before Tqrs is determined (thick line); and the value of the distal diastolic pressure is determined by extrapolating the adjusted polynomial or logarithmic function preceding the time Tfmp or Tfdd−T+Tajust (square dots). This value is defined as the extrapolated diastolic pressure value (PDE).

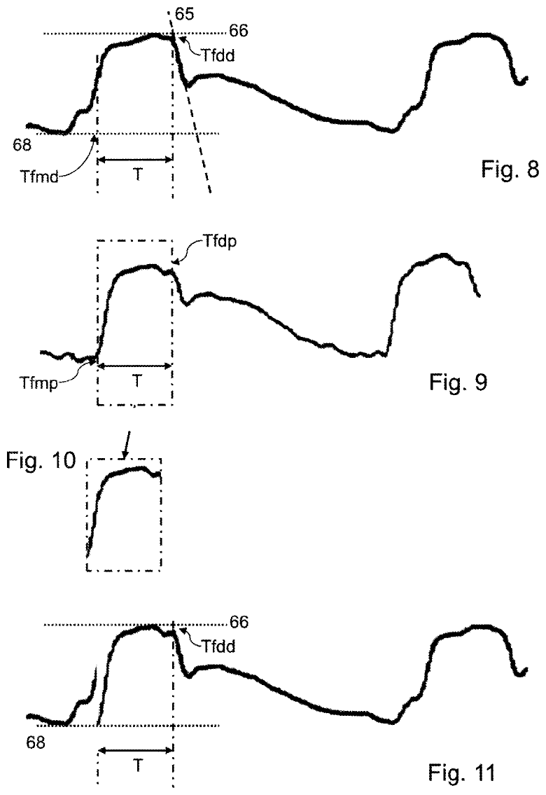

FIG. 8 illustrates an example of the processing performed on the pressure signal PSCD. In this example, the processing circuit 42 determines the tangent 65 (illustrated by a dashed line) to the pressure PSCD falling edge and the systolic pressure illustrated by the straight line 66 (illustrated by a dotted line).

The processing circuit 42 determines here the start time Tfmd of the rising edge of the pressure PSCD (or PSCD₂) by subtracting the duration T from the time Tfdd. It is also possible to envisage searching for the value Tfmd using various criteria in an interval around the value Tfdd−T. Tfmd will satisfy for example the following inequality: Tfdd−1.1*T<Tfmd<Tfdd−0.9*T, preferably Tfdd−1.05*T<Tfmd<Tfdd−0.95*T.

In a first example, if the measure of similarity between the signals PSCP₂ and PSCD₂ is high enough (the mean squared deviation is lower than a threshold or if the maximum of the autocorrelation function exceeds a threshold), the time Tfmd is calculated on the basis of the peak of the derivative of PSCD₂ that is closest to the time defined by Tfdd−T. The time Tfmd may be calculated by determining the tangent to the signal PSCD₂ at this peak of the derivative of PSCD₂, then by determining the intersection between this tangent and the extrapolated distal diastolic pressure;

In a second example, if the measure of similarity between the signals PSCP₂ and PSCD₂ is insufficient (the mean squared deviation is higher than a threshold or if the maximum of the autocorrelation function is lower than a threshold), a sufficient similarity is sought anew over only one portion of the signals of the pressures PSCP₂ and PSCD₂ or of their derivatives in the middle of the period T, preferably over a portion of between 20% and 80% of the interval T. The measure of similarity is calculated for various time offsets (for example with time increments that are for example between 0.1 milliseconds and 0.5 milliseconds (corresponding to the sampling period), over a window of ±20 milliseconds) between the signal PSCD₂ and the signal PSCP₂. The maximum measure of similarity is obtained for a value Tajust. If a sufficient measure of similarity is obtained for this portion of the signals, with this time offset Tajust, the time Tfmd is defined exactly by Tfmd=Tfdd−T+Tajust.

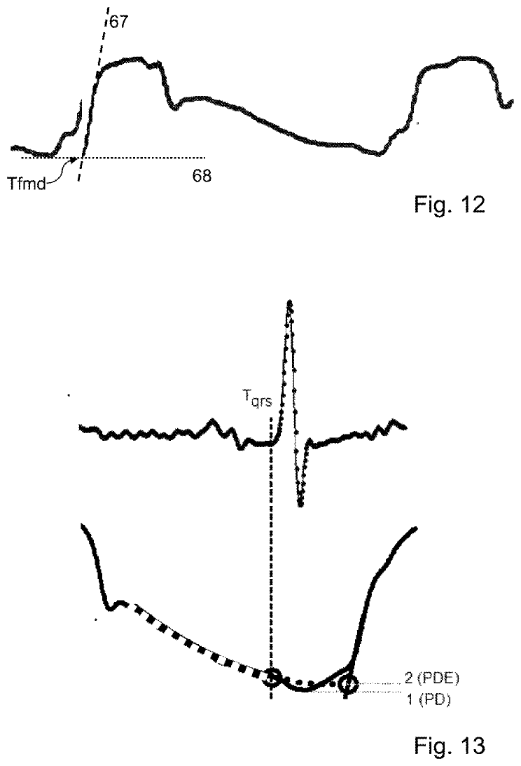

In a third example, according to another calculation method, if the measure of similarity between the signals PSCP₂ and PSCD₂ is insufficient, the signal PSCP₂ between the times Tfmp and Tfdp is retrieved, as illustrated in FIGS. 9 and 10. The retrieved signal is adapted to the scale of the signal PSCD₂, as illustrated in FIG. 11. The abscissa scale (time axis) of the retrieved signal PSCP₂ remains unchanged. The ordinate scale of the retrieved signal PSCP₂ is adapted such that its extremum corresponds to the distal systolic pressure defined by the straight line 66, and such that its minimum corresponds to the extrapolated distal diastolic pressure defined by the straight line 68. The retrieved signal PSCP₂ is adapted and inserted by replacing the signal PSCD₂ between the times Tfdd−T and Tfdd. As illustrated in FIG. 12, the time Tfmd may be determined as the intersection between the extrapolated distal diastolic pressure 68 and the tangent 67 to the rising edge of this inserted signal PSCD₂. This alternative make it possible to benefit from the accuracy of the signal PSCP₂ in order to determine the time Tfmd. Other methods for adapting a rising edge of the signal PSCP₂ to the signal PSCD₂ may also be envisaged, with a view to defining the time Tfmd.

In a fourth example, if the measure of similarity between the signals PSCP₂ and PSCD₂ is sufficient, if a second peak of the derivative of the pressure PSCD₂ is detected, and if this second peak of the derivative of the pressure PSCD₂ is closer to the time defined by (Tfdd−T) than the first peak of the derivative detected in order of increasing time, the processing circuit 42 could generate a suitable warning signal in order to attract the attention of the practitioner, since this may be associated with a coronary stenosis. Staying with this case, another parameter to be taken into account could be the amplitude of the pressure PSCD₂ corresponding to the first peak of the derivative of the pressure detected in order of increasing time and the ratio of this amplitude to the value of the pressure at the time Tqrs just before the QRS complex (see FIG. 13): when this ratio is above a threshold, the processing circuit 42 could generate a suitable warning signal in order to attract the attention of the practitioner, since this may also be associated with a coronary stenosis.

When the measure of similarity between the signals PSCP₂ and PSCD₂ is insufficient, the processing circuit 42 could generate a suitable warning signal in order to attract the attention of the practitioner, since this may be associated with a very severe coronary stenosis.

FIG. 5 is a diagram illustrating a superposition of an example of the proximal coronary blood pressure PSCP (for a left anterior descending coronary artery) over a plurality of heartbeats, with a corresponding electrocardiogram signal ECG, the derivative of the pressure PSCP, and a proximal signal SPR reconstructed on the basis of the electrocardiogram signal. The reconstructed proximal signal SPR corresponding to the bottom curve in the proximal position may be defined according to the following relation:

SPR=a constant, a triangular function or preferably the amplitude of the QRS complex of the electrocardiogram signal having a width of preferably 10-20 milliseconds and centered on the time Tfmp.

SPR=0 otherwise.

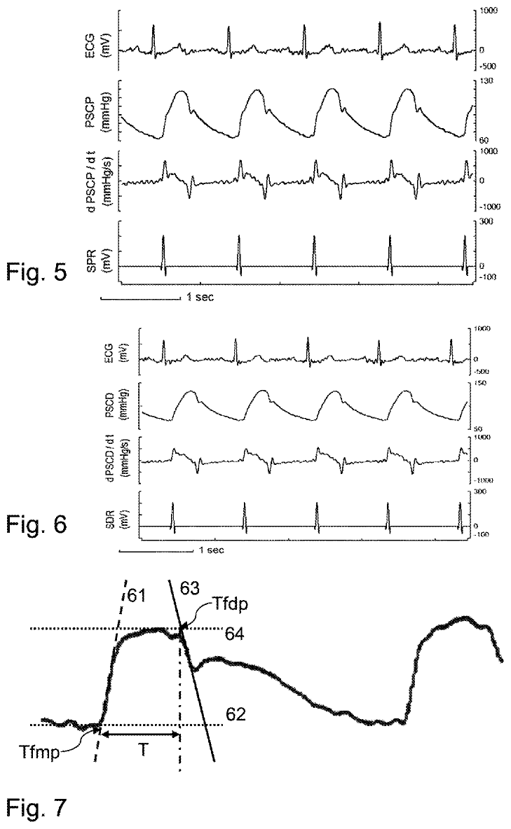

FIG. 6 is a diagram illustrating a superposition of an example of the pressure PSCD over a plurality of heartbeats, with a corresponding electrocardiogram signal ECG, the derivative of the pressure PSCD, and a distal signal SDR reconstructed on the basis of the electrocardiogram signal. The reconstructed distal signal SDR corresponding to the bottom curve in the distal position may be defined according to the following relation:

SDR=a constant, a triangular function or preferably the amplitude of the QRS complex of the electrocardiogram signal having a width of preferably 10-20 milliseconds and centered on the time Tfmd.

SDR=0 otherwise.

The time Tmd taken by the blood pressure wave to propagate through the coronary artery, between the proximal and distal measurement positions, is determined by the relationship: Tmd=(Tfmd−Tfmp) (modulo Tc), i.e. the remainder from the division of Tmd by Tc.

Tc is the mean period of a blood pulsation, in the case in which the signal PSCP (or PSCP₂) and the signal PSCD (or PSCD₂) used for the calculations are not simultaneous.

The coronary pulse wave velocity CPWV is then calculated by the following relationship:

CPWV=Dmd/Tmd

The calculated velocity CPWV may be compared with a reference threshold for a similar artery and a similar patient. When the velocity CPWV crosses such a reference threshold (a lower threshold or an upper threshold, as appropriate), the processing circuit 42 could generate a suitable warning signal in order to attract the attention of the practitioner. Various thresholds could be used, in particular depending on various risk factors, such as hypertension, diabetes, dyslipidemia, smoking, family history of coronary cardiovascular problems or a prior coronary cardiovascular episode.

In the case in which the PSCP and PSCD signals are not available at the same time, the times Tfmd and Tfmp may be determined by their respective offsets Offsprox and Offsdis with respect to the QRS complex of the electrocardiogram. For example, the autocorrelation between an electrocardiogram signal and the reconstructed signal SPR is calculated on the basis of PSCP (or PSCP₂) and the autocorrelation between an electrocardiogram signal and the reconstructed signal SDR is calculated on the basis of PSCD (or PSCD₂). An autocorrelation function of parameter k between two functions S1 and S2 typically comprises the following calculation:

Σᵢ₌₁^{N}S1(i)*S2(i+k)

The autocorrelation function may be normalized. The autocorrelation function may also be averaged over a plurality of cardiac cycles in order to limit the effect of potential dispersions due to respiration leading to substantial errors over only one cycle.

In the autocorrelation function, i is a time index of a sample of the function S1 or S2 and N is a number of samples studied. By performing this calculation for various values of the parameter k, the maximum of the autocorrelation function is obtained for the value K corresponding to the time offset between the functions S1 and S2. The value of the time offset between the functions S1 and S2 is then K*ΔT, where ΔT is the sampling period of the functions S1 and S2.

The autocorrelation functions therefore make it possible to calculate the offset Offsprox between the QRS complex of the electrocardiogram and the rising edge of the PSCP (or PSCP₂) and to calculate the time offset Offsdis between the QRS complex of the electrocardiogram and the rising edge of the PSCD (PSCD₂). The duration Tmd is deduced by Tmd=Offsdis−Offsprox.

If it is possible to calculate the autocorrelation function between the electrocardiogram and the reconstructed signals SPR and SDR over a plurality of consecutive analysis periods, it will be possible to obtain time sequences for offsets Offsprox and Offsdis, respectively. When the value of the peak of the autocorrelation function is too low (below a threshold whose value is preferably between 0.4 and 0.6) over one analysis period, this analysis period will be excluded from the time sequences of the values Offsdis or Offsprox, since this peak will correspond to a noisy electrocardiogram signal or to an erroneous detection of the times Tfmp and Tfmd.

In the case of consecutive analysis periods, time sequences for offsets Offsprox and Offsdis, respectively, may be generated. The means of these time sequences for offsets M_{offsprox} and M_{Offsdis} will be used to calculate the duration Tmd=M_{Offsdis}−M_{Offsprox}. When a linear relationship can be demonstrated between the time sequences of the offsets Offsprox and Offsdis and the time sequences of the corresponding diastolic arterial pressures (PAD), averaged over the same periods as the offsets, a correction for these offsets Offsprox and Offsdis will be made as described below in order to obtain the offsets for constant diastolic arterial pressures:
A common PAD, PADc, will be calculated between the proximal PAD and the distal PAD: preferably the mean of the proximal and distal PADs. Otherwise, one or the other of the two, proximal or distal, diastolic arterial pressures will be taken.
Linear regressions are sought in the clouds of points Offsprox with respect to the PAD in the proximal position (PADprox) according to the law: Offsprox=Kprox×PADprox+Cprox. The correlation coefficient Rprox and the probability Pprox of the regression will also be calculated.
Linear regressions are sought in the clouds of points Offsdis with respect to the PAD in the distal position (PADdis) according to the law: Offsdis=Kdis×PADdis+Cdis. The correlation coefficient Rdis and the probability Pdis of the regression will also be calculated.
The offsets Offsprox and Offsdis will be corrected in order to extrapolate their value for this common value PADc.
If linear regressions are demonstrated in both proximal and distal positions, i.e. if Rprox and Rdis are above a given threshold and/or Kprox and Kdis are above a given threshold and/or the probabilities Pprox and Pdis are below a given threshold, the corrected offsets Offsprox′ and Offsdis′ will be:

Offsprox′=Offsprox−Kprox×(PADprox−PADc)

Offsdis′=Offsdis−Kdis×(PADdis−PADc)
In the case in which a linear regression is demonstrated only in the proximal position, i.e. Rprox is above a given threshold and/or Kprox is above a given threshold and/or the probability Pprox is below a given threshold, the corrected offsets Offsprox′ will be:

Offsprox′=Offsprox−Kprox×(PADprox−PADc), with PADc=PADdis.
In the case in which a linear regression is demonstrated only in the distal position, i.e. if Rdis is above a given threshold and/or Kdis is above a given threshold and/or the probability Pdis is below a given threshold, the corrected offsets Offsdis' will be:

Offsdis'=Offsdis−Kdis×(PADdis−PADc), with PADc=PADprox.

According to another calculation method, the offsets could be corrected for in the same way as a function of systolic arterial pressures or heart rates instead of diastolic arterial pressures.

When the means of the time sequences for the pressures PADprox and PADdis reveal a decrease in the distal pressure, and when the amplitude of this decrease is greater than a given threshold, the processing circuit 42 could generate a suitable warning signal in order to attract the attention of the practitioner, since this may be associated with a coronary stenosis.

Furthermore, the processing circuit 42 could generate a warning signal for other scenarios that may be associated with a coronary stenosis. After having determined the value Tfmd, the value Tpfm=Tfmd−Tana is calculated, where Tana is a value between 5 and 10 milliseconds. Tana corresponds to an analysis duration corresponding to an artifact rise in the distal coronary blood pressure before the rising edge. The value Ptpfm of PSCD at the time Tpfm is determined. The ratio R=Ptpfm/PDE is calculated, where PDE is the extrapolated diastolic pressure at the distal position. If R exceeds a threshold (between 0.95 and 1.05 for example), the processing circuit 42 could generate a warning signal, since such a ratio R may be highly representative of the ischemic character of a coronary stenosis, without having to resort to vasodilating substances such as adenosine.

The methods for determining the times Tfmp and Tfmd described may employ searching for an intersection with a straight line that is representative of a diastolic pressure. However, the diastolic pressure may be subject to substantial variations with time. It may therefore prove to be advantageous to correct for the measured diastolic pressure values by using a low-pass filter in order to filter the rapid variations in the signal PSCP or PSCD (or PSCP₂ and PSCD₂).

Using an FFR guide wire, the use of which has been approved by the health authorities and forms part of routine clinical practice, makes it possible to use a system as described herein with a substantially streamlined clinical validation process.

In the example described in detail above, the FFR guide wire 3 includes two pressure sensors 31, 32 that are separated from each other by a fixed predetermined distance Dmd. It is also possible to envisage using an FFR guide wire fitted with a single pressure sensor, which is moved by the practitioner over a predefined distance between the distal position and the proximal position in the coronary artery. During the analysis of the respective measurement signals at the proximal position and at the distal position, this distance Dmd is taken into account for calculating the pulse wave velocity.

It is possible for example to envisage sampling a distal coronary pressure and/or a proximal coronary pressure at a frequency of between 200 Hz and 2 kHz. For a sampling frequency that is deemed insufficient, it is possible to interpolate the sampling values (for example using cubic splines), then to sample the interpolated signal anew at a frequency higher than the initial sampling frequency (oversampling). For example, for a sampling frequency of 500 Hz, it is possible to envisage oversampling the interpolated signal at a frequency of 2 kHz or more.

Tests were carried out on a sample of 59 patients using a system for determining the coronary pulse wave velocity as described herein. These tests were carried out on patients that had undergone coronary angiograms and been recommended for FFR analysis. The recommendation for FFR analysis followed in particular a visual examination noting a decrease in diameter of more than 50% at the site of a stenosis in the left descending coronary artery (LDA), in the right coronary artery (RCA) or in the circumflex coronary artery (Cx).

The FFR analyses were carried out using pressure measurement guide wires sold under the reference “C12058” by St. Jude Medical. The invasive blood pressure was measured using these guide wires and sampled on a computer by means of an analog/digital acquisition card at a frequency of 500. Electrocardiogram signals were measured and sampled at the same time. Blood pressure and electrocardiogram signals were also measured at the same time at the sinus of Valsalva, at the brachiocephalic arterial trunk and at the radial artery. In order to determine the distance between the sites at which the distal and proximal pressures were measured, this distance was determined according to the movement of the outer portion of the guide wire.

By denoting the coronary pulse wave velocity by the value CPWV, a mean value of CPWV of 10.37 (±6.23) meters per second was measured for these patients with excellent reproducibility. More specifically, a mean value of CPWV of 10.06 (±4.85) meters per second was measured for the left anterior interventricular coronary arteries, a mean value of CPWV of 10.07 (±6.86) meters per second was measured for the right coronary arteries, and a mean value of CPWV of 12.4 (±7.06) meters per second was measured for the circumflex coronary arteries. It was observed that the CPWV value was higher for those coronary arteries including a stent 14.25 (±5.68) meters per second. Furthermore, for one and the same coronary artery, the insertion of a stent led to an increase in the CPWV from 9.79 (±5.1) meters per second to 17.34 (±9.03) meters per second, which mechanically speaking might be expected when a metal element is implanted within an elastic structure. The determinants of the CPWV were kidney function, body mass index and the presence of a stent.

Furthermore, a significant correlation between the aortic pulse wave velocity values measured invasively and non-invasively was observed. These observations make it possible to conclude that the CPWV values determined using a system as described herein are reliable.

A system as described herein may prove to be particularly advantageous for the detection of intermediate stenoses in coronary arteries as such stenoses are generally undetectable, in particular in young patients.

1-14. (canceled)

15. An apparatus for determining a velocity of a coronary pulse wave velocity, said apparatus comprising an interface and a processing device, wherein said interface is configured to receive a proximal signal, and a distal signal, wherein said proximal signal is a proximal blood pressure signal and said distal signal is a distal blood pressure signal, said proximal and distal signals being indicative of pressure in a coronary artery, wherein said processing device is configured to identify, based on said proximal signal, a first start time, a second start time, and a first duration between said first and second start times, wherein said first start time is a start time of a rising edge of said proximal signal and said second start time is a start time of a falling edge of said proximal signal, wherein said processing device is configured to identify, based on said distal signal, a third start time, a fourth start time, wherein said third start time is a start time of a rising edge of said distal signal and said fourth start time is a start time of a falling edge of said distal signal, wherein said processing device is configured to retrieve a distance between a location at which said proximal signal is measured and a location at which said distal signal is measured, wherein said processing device is configured to determine a second duration, wherein said second duration separates said first start time and said third start time, and wherein said processing device is configured to determine said velocity based on said second duration and said distance.

16. The apparatus of claim 15, wherein said processing device is further configured to calculate a derivative of said distal signal and to identify said fourth start time as corresponding to a time at which said derivative has a peak that is closest to a time that precedes said third start time by said first duration.

17. The apparatus of claim 15, wherein said processing device is further configured to derive a distal blood pressure signal by subtracting a distal diastolic pressure from said distal signal and to derive a proximal blood pressure signal by subtracting a proximal diastolic pressure from said proximal signal, wherein said processing device is further configured to synchronize falling edges of said distal blood pressure signal and said proximal blood pressure signal and to determine a measure of similarity between said distal and proximal blood pressure signals over the course of said first duration.

18. The apparatus of claim 17, wherein if said measure of similarity is insufficient, said processing device is configured to retrieve a proximal blood pressure signal between said first start time and said second start time, to adapt said retrieved signal to a scale of said distal signal, to replace an interval of said distal signal with said retrieved and adapted signal, and to determine said fourth start time as being an intersection between a tangent to said adapted retrieved signal and a distal diastolic pressure, wherein said interval in which said distal signal is replaced by said retrieved and adapted signal is an interval that is ends at said third start time and begins at a time that is earlier than said third start time by said first duration.

19. The apparatus of claim 17, wherein if said measure of similarity is insufficient, said processing device is configured to calculate a measure of similarity between said distal blood pressure signal and said proximal blood pressure signal over an interval that is between two tenths of said first duration and eight tenths of said first duration around said synchronized falling edges at various time offsets and to determine which of said offsets corresponds to a maximum measure of similarity between said distal blood pressure signal and said proximal blood pressure signal, and wherein said processing device is configured to determine that said fourth start time is a difference between said third start time and an adjusted first duration, said adjusted first duration corresponding to said first duration offset by said offset that corresponds to said maximum measure of similarity.

20. The apparatus of claim 15, wherein said processing device is further configured to determine said fourth start time as being within an interval that begins at a time that precedes said third start time by an amount that is 10% greater than said first duration and that ends at a time that precedes said third start time by an amount that is 90% of said first duration.

21. The apparatus of claim 15, wherein said processing device is configured to determine said fourth start time to be an intersection between a distal diastolic pressure signal and a tangent to said rising edge of said distal signal.

22. The apparatus of claim 21, wherein said processing device is further configured to determine a third duration and a fourth duration, wherein said third duration separates a QRS complex from a received electrocardiogram signal from said first start time and wherein said fourth duration separates said QRS complex from said fourth start time, wherein said processing device is configured to create first and second reconstructed signals and to determine first and second intercorrelation functions, wherein said first reconstructed signal is equal to zero everywhere except for an interval having a width of between ten and twenty milliseconds that is centered on a time that corresponds to said first start time, wherein during said interval, said first reconstructed signal is one of constant, a triangular function, and an amplitude of said QRS complex, wherein said second reconstructed signal is equal to zero everywhere except for an interval having a width of between ten and twenty milliseconds that is centered on a time corresponding to said fourth start time, wherein during said interval, said reconstructed signal is equal to one of a constant, a triangular function, and an amplitude of said QRS complex, wherein said first intercorrelation function is an intercorrelation between said electrocardiogram signal and said first reconstructed signal over a duration of said cycle, wherein said processing device is further configured to calculate a mean over said cycle of said third and fourth durations on the basis of an offset between said electrocardiogram and said first and second reconstructed signals for which said interrelation function is maximum if and only if said maximum is above a threshold that is between 0.4 and 0.6.

23. The apparatus of claim 15, wherein said interface is configured to receive an electrocardiogram signal having a QRS complex, wherein said processing device is configured to determine a duration that separates said QRS complex from said first start time, and wherein said processing device is configured to determine a duration that separates said QRS complex from said fourth start time.

24. The apparatus of claim 15, wherein said processing device is further configured to determine a distal diastolic pressure by calculating an intersection between a function and a tangent to a rising edge of said distal pressure, wherein said function is selected from the group consisting of a polynomial function and a logarithmic function, and wherein said processing device is configured to adjust said function for said distal pressure signal before a start time of a QRS complex present in an electrocardiogram received by said interface and to extrapolate after said start time.

25. The apparatus of claim 15, wherein said processing device is configured to determine said first start time as being an intersection between a tangent to said rising edge of said proximal signal and a proximal diastolic pressure and wherein said processing device is configured to determine said second start time as being at an intersection between a tangent of said falling edge of said proximal signal and a proximal systolic pressure.

26. The apparatus of claim 15, wherein said processing device is configured to determine a value of said distal signal at a time that precedes said fourth start time by between five and ten milliseconds, to determine a ratio of said value to a distal diastolic blood pressure, and to generate a warning signal if said ratio is higher than a threshold of between 0.95 and 1.05.

27. The apparatus of claim 15, further comprising an elongated FFR guide wire, a proximal pressure sensor, and a distal pressure sensor, wherein said proximal and distal pressure sensors are separated by a predefined distance along said guide wire, wherein said proximal and distal pressure sensors connect to said interface, and wherein said interface comprises a sampling circuit that samples said proximal and distal signals from said first and second pressure sensors.

28. The apparatus of claim 27, wherein said predefined distance is between seventy millimeters and one hundred and fifty millimeters.
